(19) United States
(12) Patent Application Publication
(10) Pub. No.: US 2023/0103221 A1
(43) Pub. Date: Mar. 30, 2023

(54) IMPLANTABLE MEDICAL DEVICE WITH WINDOW FOR WIRELESS POWER TRANSFER

(71) Applicant: Cardiac Pacemakers, Inc., St. Paul, MN (US)

(72) Inventors: Jean M. Bobgan, Maple Grove, MN (US); Joey Chen, Stevenson Ranch, CA (US); James M. English, Cahir (IE); Keith R. Maile, New Brighton, MN (US); Brian T. Swanson, Fridley, MN (US)

(21) Appl. No.: 17/951,561

(22) Filed: Sep. 23, 2022

Related U.S. Application Data
(60) Provisional application No. 63/247,897, filed on Sep. 24, 2021.

(51) Int. Cl. A61N 1/378; H02J 50/20; H02J 50/90

(52) U.S. Cl. CPC A61N 1/3787; H02J 50/20; H02J 50/90

(57) ABSTRACT

An implantable medical device comprises a hermetically sealed housing including at least a first window configured for wireless transfer of an external power signal therethrough, an antenna disposed within the housing at a position such that the antenna can receive the external power signal through the window, and circuitry disposed within the housing and operatively coupled to the antenna.

### CROSS REFERENCE TO RELATED APPLICATION

This application claims priority to U.S. Provisional Application No. 63/247,897, filed Sep. 24, 2021, which is incorporated herein by reference.

### TECHNICAL FIELD

The present invention relates to medical devices for sensing physiological parameters and/or delivering therapy. More specifically, the invention relates to devices and methods for recharging implantable medical devices that may be used to sense physiological parameters and/or deliver therapy.

### BACKGROUND

Implantable medical devices (IMDs) may be configured to monitor physiological parameters, deliver signals, and/or provide therapy. Implantable medical devices require a source of charge for performing these functions which may be provided through a battery or an alternate external power supply.

### SUMMARY

In Example 1, an implantable medical device comprising a hermetically sealed housing, an antenna and circuitry. The housing defines an interior chamber and includes at least one window configured for wireless transfer of an external power signal to the interior chamber. The antenna is disposed within the interior chamber at a position such that the antenna can receive the external power signal through the window. The circuitry is disposed within the interior chamber and is operatively coupled to the antenna.

In Example 2, the implantable medical device of Example 1, wherein the housing comprises first and second side walls and a peripheral wall extending therebetween, and wherein the at least one window forms a portion of the first side wall.

In Example 3, the implantable medical device of either of Examples 1 or 2, wherein the at least one window includes first and second windows disposed on opposite sides of the housing, and wherein the antenna is disposed within the interior chamber at a position such that the antenna can receive an external power signal through the first window.

In Example 4, the implantable medical device of Example 3, wherein the housing includes first and second side walls and a peripheral wall extending therebetween, and wherein the first window forms a portion of the first side wall, and the second window forms a portion of the second side wall, and further wherein the antenna is disposed within the interior chamber at a position such that the antenna can receive an external power signal through the first window.

In Example 5, the implantable device of Example 4, further comprising a braze ring positioned between the at least window and the first side wall.

In Example 6, the implantable device of Example 1, wherein the at least one window has one of a circular, semi-circular, and ovular shape.

In Example 7, the implantable device of Example 1, wherein the at least one window is formed of a non-metallic material.

In Example 8, the implantable device of Example 8, wherein the at least one window is formed from a ceramic.

In Example 9, the implantable medical device of any of Examples 1-8, wherein the antenna is configured as a planar antenna.

In Example 10, the implantable medical device of any of Examples 1-9, wherein the interior chamber comprises a battery operatively coupled to the antenna and further operatively coupled to the circuitry for providing power thereto.

In Example 11, the implantable medical device of any of Examples 1-9, wherein the antenna receives the external power signal through the at least one window to directly power the circuity of the implantable medical device.

In Example 12, the implantable medical device of any of Example 1 -11, wherein the antenna is positioned adjacent a ferrite layer.

In Example 13, the implantable medical device of Example 12, wherein the ferrite layer is positioned adjacent a copper layer.

In Example 14, the implantable medical device of any of Examples 1-11, wherein the antenna is positioned adjacent a copper layer.

In Example 15, the implantable medical device of any of Examples 1-14, wherein the device further comprises a signal antenna for receiving and transmitting a radio frequency signal.

In Example 16, an implantable medical device comprising a hermetically sealed housing, an antenna and circuitry. The housing includes at least a first window configured for wireless transfer of an external power signal therethrough. The antenna is disposed within the housing at a position such that the antenna can receive the external power signal through the window. The circuitry is disposed within the housing and is operatively coupled to the antenna.

In Example 17, the implantable medical device of Example 16, wherein the housing comprises first and second side walls and a peripheral wall extending therebetween, and wherein the first window forms a portion of the first side wall.

In Example 18, the implantable device of Example 17, further comprising a braze ring positioned between the first window and the first side wall.

In Example 19, the implantable medical device of Example 16, wherein the housing further includes a second window, wherein the first and second windows are disposed on opposite sides of the housing, and wherein the antenna is disposed within the housing at a position such that the antenna can receive an external power signal through first window.

In Example 20, the implantable medical device of Example 19, wherein the housing includes first and second side walls and a peripheral wall extending therebetween, and wherein the first window forms a portion of the first side wall, and the second window forms a portion of the second side wall, and further wherein the antenna is disposed within housing at a position such that the antenna can receive an external power signal through the first window.

In Example 21, the implantable device of Example 16, wherein the first window has one of a circular, semi-circular, and ovular shape.

In Example 22, the implantable device of Example 16, wherein the first window is formed of a non-metallic material.

In Example 23, the implantable device of Example 22, wherein the first window is formed from a ceramic.

In Example 24, the implantable medical device of Example 16, wherein the antenna is configured as a planar antenna.

In Example 25, the implantable medical device of Example 16, further comprising a battery operatively coupled to the antenna and further operatively coupled to the circuitry for providing power thereto.

In Example 26, the implantable medical device of Example 16, wherein the antenna receives the external power signal through the window to directly power the circuity of the implantable medical device.

In Example 27, the implantable medical device of Example 16, wherein the antenna is positioned adjacent a ferrite layer.

In Example 28, the implantable medical device of Example 27, wherein the ferrite layer is positioned adjacent a copper layer.

In Example 29, the implantable medical device of Example 16, wherein the antenna is positioned adjacent a copper layer.

In Example 30, an implantable medical device comprising a hermetically sealed housing, a first antenna, a second antenna and circuitry. The housing defines an interior chamber and includes at least one window configured for signal transfer between an external device and the interior chamber. The first antenna is disposed within the interior chamber at a position such that the first antenna can receive an external power signal through the at least one window. The second antenna is disposed within the interior chamber at a position such that the second antenna can receive and transmit signals through the at least one window. The circuitry is disposed within the interior chamber and is operatively coupled to the first antenna.

In Example 31, the implantable medical device of Example 30, wherein the housing comprises first and second side walls and a peripheral wall extending therebetween, and wherein the first window forms a portion of the first side wall.

In Example 32, the implantable medical device of Example 32, wherein the at least one window further includes a second window forming a portion of the second side wall.

In Example 33, a method of making an implantable medical device, the method comprising forming a housing having a first side wall, a second side wall and a peripheral wall therebetween, wherein the first side wall includes a window configured for wireless transfer of an external power signal therethrough, positioning an antenna within the housing such that the antenna can receive the external power signal through the window, and positioning circuitry within the housing, the circuitry operatively coupled to the antenna.

In Example 34, the method of Example 33, further comprising positioning a battery within the housing, the battery being operatively coupled to the antenna and further operatively coupled to the circuitry for providing power thereto.

In Example 35, the method of Example 33, wherein the antenna is configured to receive the external power signal through the window to directly power the circuity of the implantable medical device.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows an exemplary implantable medical device (IMD) that can be used in relation to embodiments of the present invention,

FIG. 2 shows an exploded view of the exemplary IMD of FIG. 1,

FIG. 3 shows a front view of an antenna that can be used in relation to embodiments of the present invention,

FIG. 4 shows a portion of the IMD of FIG. 1,

FIG. 5 shows an exploded view of an exemplary IMD that can be used in relation to embodiments of the present embodiment,

FIG. 6A is an exemplary implantable medical device that can be used in relation to embodiments of the present invention,

FIG. 6B is an exemplary implantable medical device that can be used in relation to embodiments of the present invention,

FIG. 6C is an exemplary implantable medical device that can be used in relation to embodiments of the present invention,

FIG. 6D is an exemplary implantable medical device that can be used in relation to embodiments of the present invention, and

FIG. 6E is an exemplary implantable medical device that can be used in relation to embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

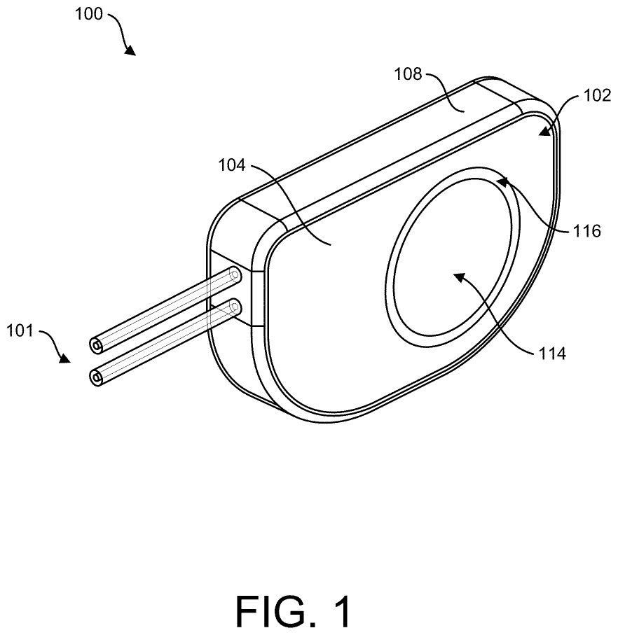

FIG. 1 illustrates an implantable medical device (IMD) 100 for implanting into a patient. In embodiments, the IMD 100 may be implanted subcutaneously within an implantation location or pocket in the patient and may be configured to provide therapy to target patient tissue and/or to monitor (e.g., sense and/or record) physiological parameters associated with the patient. In embodiments, the IMD 100 may also be configured for receiving and/or transmitting signals from the patient and/or from external devices. The IMD 100 may be configured for delivering therapy and/or monitoring, receiving or delivering signals at regular intervals, continuously, and/or in response to a detected event. In various embodiments, a detected event may be detected by one or more sensors of the IMD 100, another IMD (not shown), an external device (not shown), an/or the like. As such, the IMD 100 may be configured to detect a variety of physiological signals that may be used in connection with various diagnostics, therapeutic, and or monitoring implementations. In embodiments, the IMD 100 may be used in urological, neurological, cardiac, or any other appliable field that uses an implantable medical device for receiving and/or transmitting signals. The IMD 100 requires charge and/or power signals to be provided for maintained proper function of IMD 100. Various embodiments of IMD 100 directed towards receiving and transmitting power and various other signals will be described herein.

In exemplary embodiments, the IMD 100 may be configured as a urological therapy device to deliver selective stimulation to, for example, the sacral nerves for treatment of urological disorders such, without limitation, bladder and/or bowel control disorders. In other embodiments, the IMD 100 may be configured as a neurostimulation therapy device for pain management and the like. In still other embodiments, the IMD 100 may be a cardiac rhythm management (CRM) device for sensing and stimulating cardiac tissue for treatment of cardiac arrhythmias such as bradycardia, tachycardia and for cardiac resynchronization therapy. In other embodiments, the IMD 100 may be configured to deliver a fluid to a target tissue or organ. In still other embodiments, the IMD 100 may be configured as a monitoring device only, with no therapeutic functionality, to monitor physiological parameters of a patient. In short, the present disclosure is not limited to any particular clinical application, and any implantable device that requires power to operate as intended.

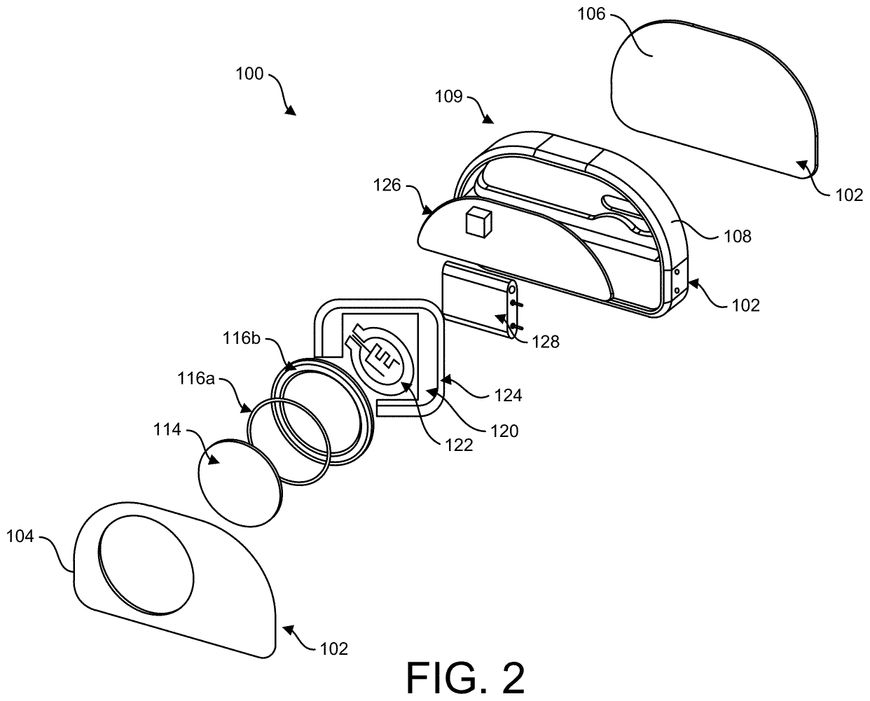

As shown in FIG. 1, the IMD 100 includes a hermetically sealed housing 102 having a first side wall 104, a second side wall 106 (FIG. 2), and a peripheral wall 108 extending between the first and second side walls 104, 106. In embodiments, the housing 102 of the IMD 100 defines an interior chamber defined by the space within the IMD 100 that is bordered by the first side wall 104, the second side wall 106 and the peripheral wall 108. In some instances, the housing 102, and thus the first side wall 104, the second side wall 106 and the peripheral wall 108, are composed of a metallic material, such as, but not limited to, titanium. As shown, the IMD 100 additionally includes at least one window 114. In the various embodiments, the window 114 forms part of the housing 102.

In embodiments, the window 114 may be composed of a hermetic and/or non-conductive material. The window 114 may be composed of materials such as, but not limited to, Cermet, alumina, and sapphire. In various embodiments, the window 114 forms at least a portion of the first side wall 104 of the housing 102. Although illustrated with the window 114 defining at least a portion of the first side wall 104, the window 114 may define a portion of the second side wall 106 and/or the peripheral wall 108. In various embodiments, the window 114 is configured for allowing transmittal of power signals and/or other signals from external devices to the components housed within the IMD 100 and vice versa. For example, in some embodiments, an external power source is used in combination with the window 114 of the IMD 100 to pass power signals generated by the external power source (not shown) through the window 114 and into the interior chamber of the housing 102 and then to an antenna 122 (see FIG. 2) of the IMD 100, and thus provide energy to recharge the implantable medical device 100, as will be described further herein.

In embodiments, the at least one ring 116 is configured for hermetically sealing the window 114 to first side wall 104 as illustrated in FIG. 2. As previously mentioned, in other embodiments, the ring 116 may provide a hermetic seal between the window 114 to the second wall 106 (FIG. 2) and/or to the peripheral wall 108. In various embodiments, the at least one ring 116 is a brazed alloy, composed of materials such as, but not limited to, gold and copper. In embodiments wherein the at least one ring 116 provides a hermetic seal between window 114 and housing 102, instances of leakage of external materials into the interior chamber of the housing 102 may be reduced. While FIG. 1 illustrates the at least one ring 116 comprising a first, singular ring 116, the ring 116 may comprise a plurality of rings 116 for sealing the window 114, as will be described further with reference to FIG. 2. Additionally, while illustrated as generally circular, the at least one ring 116 may comprise variations in shape based on the configuration of the window 114 such that regardless of the shape or size of the window 114, the at least one ring 116 is configured for facilitating a hermetic sealing between the window 114 and the housing 102. The IMD 100 may additionally include a plurality of sensors 101. The sensors 101 may be signaling, sensing, and/or stimulating sensors, depending on the intended use of the IMD 100.

FIG. 2 is an exploded view of the IMD 100 of FIG. 1. As illustrated, the housing 102 includes the first side wall 104 positioned opposite the second side wall 106. The peripheral wall 108 of the housing 102 is illustrated surrounding a housing body 109. The first side wall 104 includes an opening for receiving the window 114 with the at least one ring 116 configured for surrounding the window 114 and providing a hermetic sealing between the window 114 and the housing 102, as previously described with reference to FIG. 1. In the illustrative embodiment of FIG. 2, the at least one ring 116 includes the first ring 116a and a second ring 116b. In embodiments, the first ring 116a is composed of braze material, as discussed above, and the second ring 116b is a pre-formed braze ring configured to facilitate formation of the hermetic brazed seal between the window 114 and the adjacent surface defining the opening in the side wall 106. Because FIG. 2 depicts a simplified exploded view of the IMD 100, the first ring 116a is depicted as a discrete component, although the skilled artisan will readily recognize that the first ring 116a is, in practice, formed in situ during a brazing process as is known in the art. In the various embodiments, the technique for securing and hermetically sealing the window 114 in the opening in the side wall 106 is not limited to any particular structure or process, and the skilled artisan will recognize that any suitable technique and structure(s) for accomplishing these functions can be employed within the scope of the present disclosure.

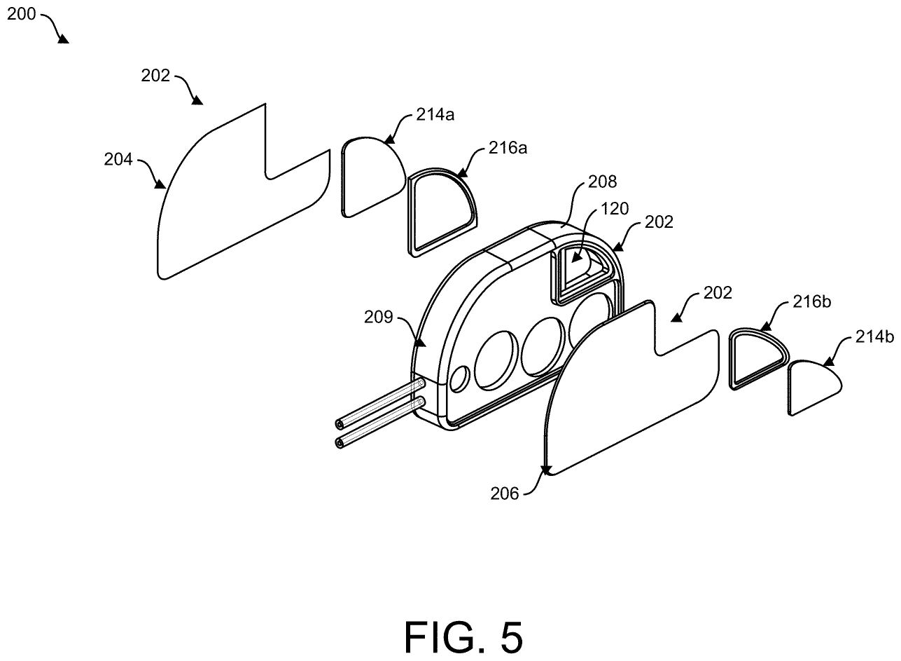

In the illustrative embodiment of FIG. 2, the IMD 100, and specifically the interior chamber defined by the housing 102, further includes various circuitry including a first printed circuit board 120 that comprises, among other things, an antenna 122, and additional circuitry, e.g., a second printed circuit board 126. The first printed circuit board 120 and second printed circuit board 126 may each be formed through conventionally used and known methods and may incorporate various components such as resisters, transistors, etc. The antenna 122 is configured for receiving and transmitting power signals, as will be described further herein. Accordingly, it is emphasized that the term “antenna” as used herein is intended to encompass any electrical component capable of wirelessly receiving and/or transmitting electrical or electromagnetic signals or energy. In various embodiments, for example as illustrated in FIG. 2, the first printed circuit board 120 includes a ferrite layer 124 and/or a copper layer 130 which may increase the efficiency of signal transfer between an external device and the antenna 122, as will be described further with reference to FIG. 4. In embodiments, the second printed circuit board 126 includes the requisite circuitry and related components for carrying out the therapeutic and diagnostic functions of the IMD 100. The specific design of the second printed circuit board 126 is not of critical importance to the present disclosure, and thus may vary as required depending on the particular clinical need for which the IMD 100 is intended.

Additionally, in embodiments, the IMD 100 may comprise a battery 128 configured to provide power to the second printed circuit board 126 of the IMD 100. In various embodiments, the battery 128 is a rechargeable battery 128 that may be recharged via a power signal transmitted to the antenna 122 from an external power source.

The first printed circuit board 120 and the antenna 122 may be positioned and configured for receiving a power signal from external power sources through the antenna 122 to directly power the circuitry on the second printed circuit board 126. As a result, in various embodiments, the battery 128 may be omitted from the IMD 100, which can be powered directly from the external power source through the antenna 122.

As previously described with reference to FIG. 1, the window 114 is configured for allowing the transmission of power signals through the window 114 such that the external power signals are transmitted to the antenna 122. In these embodiments, the antenna 122 may be positioned within or directly adjacent the window 114 for optimal power signal transfer through the reduction of a distance between the external power source and the antenna 122. In various embodiments, the IMD 100 may thus be recharged through power transmission from external power sources to the antenna 122 through the window 114 of the housing 102.

In various embodiments, the IMD 100 may comprise various additional antennae, for example a second, signal antenna 123 configured for receiving and transmitting signals such as, but not limited to, radio frequency (RF) signals. In the illustrated embodiment, the signal antenna 123 is also positioned on the first printed circuit board 120. In embodiments, positioning of the first printed circuit board 120 and/or second printed circuit board 126 adjacent the window 114 to minimize the distance between the tissue and components of the first and second printed circuit boards 120, 126, such as the signal antenna 123, may increase the ability of the signal antenna 123 to efficiently and accurately receive and transmit signals from the patent tissue. This may be beneficial in embodiments wherein the IMD 100 is configured for receiving physiological signals from the patient's body and transmitting the signals to an external device, for example, diagnostic devices operated by the patient or the physician. Similarly, eliminating the need for an additional region, such as the header region previously referenced and conventionally used for implantable devices, for incorporating the antenna 122 reduces the distance between the antenna 122 and components within the implantable medical device 100. In these configurations, feed-thrus, which may otherwise be required for connecting the antenna 122 and various other components of the IMD 100, may be omitted since antenna 122 is positioned within the interior chamber of the housing 102 along with the various other components. Additionally, positioning of the antenna 122 and/or signal antenna 123 adjacent to the window 114 which is composed of a non-conductive and hermetic material in certain examples, the window 114 may minimize signal interference compared to conventional implantable devices.

By incorporating the window 114 having a capability for transmitting power to components within the IMD 100 and positioning the antenna 122 or the signal antenna 123 adjacent and/or within the window 114, a header that may otherwise be incorporated for supporting and providing connections to power for the components within housing 102 may be eliminated. In these embodiments, the elimination of an additional header is beneficial for at least the purpose of reducing the overall material and space used by IMD 100. An antenna, or various other components configured for power transmittal, may be positioned directly within window 114 forming a portion of housing 102 of IMD 100, and therefore reduce the overall space within IMD 100 that is required for transmitting power. Additionally, connections from components within the IMD 100 to an outer antenna or power source that may otherwise be required can be eliminated.

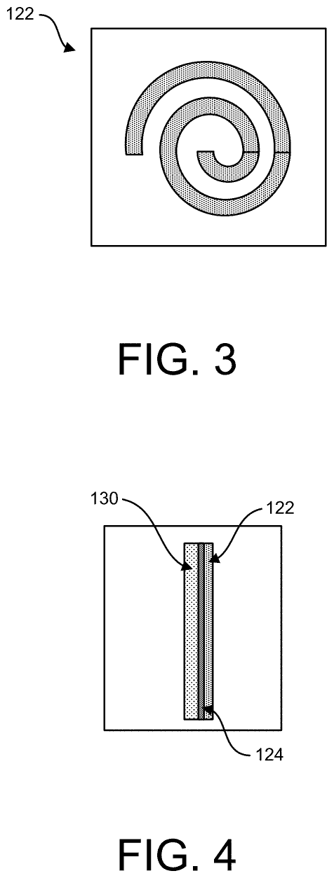

FIG. 3 is an additional view of the antenna 122 of FIG. 2. As illustrated, the antenna 122 includes a generally coiled and/or spiral shape and has a planar configuration. In various embodiments, for example as shown in FIG. 2, the antenna 122 may be generally circular. In other embodiments, the antenna 122 may include various other shapes and configurations, e.g., triangular, rectangular, or elliptical. As previously described, the antenna 122 is configured for receiving power signals from an external power source for providing charging power the battery 128 (when present) or for directly powering the implantable medical device 100. The antenna 122 may be composed of a conductive material, such as but not limited to, copper or gold. Various other appropriate conductive materials, such as metals, may be used as well. In various embodiments, the antenna 122 may be coated with a dielectric material. In some embodiments, as will be described further with reference to FIG. 4., it may be beneficial to use the antenna 122 in combination with the ferrite layer 124 (FIG. 2) and/or a copper layer 130 (FIG. 4).

FIG. 4 is a simplified cross-sectional schematic illustration of the first printed circuit board 120 illustrating the stack-up of various functional layers thereof. As illustrated in FIG. 4, the antenna 122 is positioned on the first printed circuit board 120 and adjacent the ferrite layer 124, and the copper layer 130 is positioned adjacent the ferrite layer 124 on an opposing side of the ferrite layer 124 relative to the antenna 122. In other instances, the ferrite layer 124 may be positioned adjacent the antenna 122 and the copper layer 130 may be omitted. In further examples, the copper layer 130 is positioned adjacent the antenna 122 and the ferrite layer 124 is omitted. In embodiments, the ferrite layer 124 may be a flexible sintered ferrite sheet layer, although various types of ferrite or configurations of a ferrite layer may be incorporated.

In various instances, the positioning of the ferrite layer 124 adjacent the antenna 122 is beneficial for at least the ability to focus the magnetic fields received during transmittal of power signals. For example, the ferrite layer 124 positioned adjacent the antenna 122 is capable of increasing mutual coupling between the antenna 122 and the external power signal transmitter that is transmitting power signals to the antenna 122. Additionally, positioning the ferrite layer 124 adjacent the antenna 122 may increase self-inductance within the components of the interior chamber, such as the first printed circuit board 120 and the antenna 122. In further embodiments, incorporating the ferrite layer 124 to focus the magnetic fields avoids an increase of temperature within the housing 102 of the IMD 100, which can reduce damage to the IMD 100 that would result from high temperatures, for example melting or deformation of the IMD 100. In various embodiments, this reduction of temperature is a result of eliminating the instances of eddy currents that may go around the window 114 and the IMD 100 entirely, rather than just to the window 114. In these instances, the incorporation of the ferrite layer 124 increases the efficiency of the power transfer to the antenna 122. For example, the efficiency of the power transfer between the external power device and the antenna 122 can have an efficiency value percentage of at least 70%, defined by the ratio of power signals transmitted by the external power device to the amount of power signals subsequently transmitted by the antenna 122.

For example, in one embodiment, a 1×7 coiled antenna 122 composed of copper and backed with the ferrite layer 124 was coupled with the printed circuit board 120 and coupled within a grade 1 Titanium housing 102. The frequency of signals applied was 6.78 MHz. The resulting efficiency percentage of power transfer from the external power signal to the antenna 122 was 74.4%. In similar embodiments, wherein the ferrite layer 124 was not incorporated, the efficiency percentage of the power transfer was 3.38%.

As previously described, in various instances, the copper layer 130 is positioned adjacent the ferrite layer 124 and/or the antenna 122. The copper layer 130 acts as a backing that provides a Faraday shield during operation. In other words, the copper layer 130 functions to block magnetic fields produced from transmitted of signals between the external power device and the antenna 122 from affecting the IMD 100 entirely and its various components. For example, the copper layer 130 may be beneficial for providing protection, or a shield, to the battery 128 from excess magnetic fields.

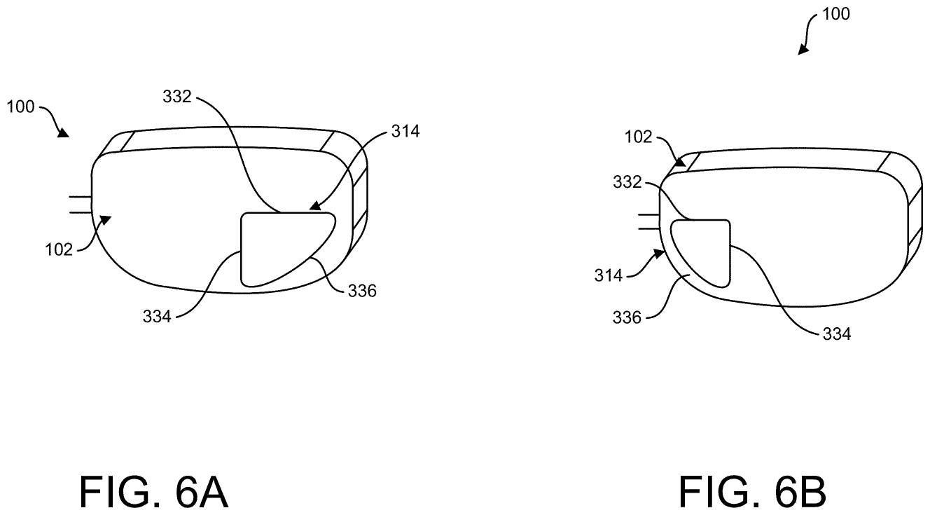

While the embodiments described herein with reference to FIGS. 1-4 were largely with reference to the use of at least one window 114, and illustratively the single window 114, the at least one window 114 may comprise two or more windows. For example, FIG. 5 illustrates an additional embodiment of an IMD 200. In embodiments, the IMD 200 of FIG. 2 may be similar to the IMD 100 as shown in FIGS. 1-4. Further, the IMD 200 may comprise the same, or similar, components as those comprised by the IMD 100 of FIG. 2.

As illustrated in FIG. 5, IMD 200 includes a housing 202 composed of a first side wall 204, a second side wall 206, a peripheral wall 208 and a housing body 209. As shown, the IMD 200 may comprise a first window 214a surrounded by a first ring 216a and positioned on the first side wall 204. The IMD 200 may additionally comprise a second window 214b surrounded by a second ring 216b and positioned on the second side wall 206. As such, in these embodiments, implantable medical device 200 is configured such that power signals are transmitted through first window 214a and power signals may be transmitted through second window 214b. First and second windows 214a, 214b may also be configured for allowing the reception or transmittal of signals other than power signals, such as telemetry and signals related to physiological signals of the patient, similar to as described with reference to the IMD 100 of FIGS. 1 and 2.

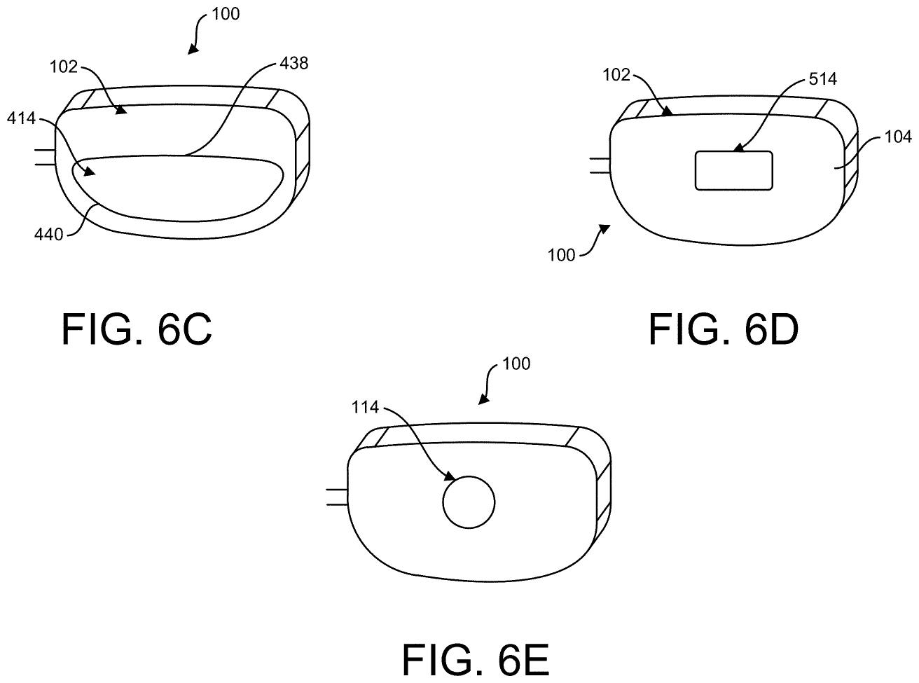

As shown in FIG. 5, IMD 200 may include the first printed circuit board 120 as described relative to the IMD 100 of FIGS. 1-4. In various instances, the IMD 200 additionally includes the second printed circuit board 216 (FIG. 2). The first and second windows 214a, 214b are illustrated with as having a wedge, or quadrant, shape as opposed to the circular shape of window 114 illustrated in FIGS. 1-2. Various other shapes and configurations of windows 114, 214 may be incorporated, as will be described further with reference to the non-limiting examples of FIGS. 6A-6E. While described as having variations, the implantable medical devices of the illustrative embodiments of FIGS. 6A-6E may be similar, or the same, in composition and function to the window 114 of IMD 100 of FIGS. 1-4 or the implantable medical device 200 of FIG. 5.

FIG. 6A illustrates implantable medical device 100 having a window 314 with a configuration comprising a wedge shape, for example resembling a quadrant of a circle. In other words, window 314 includes a first linear edge 332 and a second linear edge 334 and an arcuate portion 336 extending between the first and second linear edges 332, 334. FIG. 6B illustrates the implantable medical device 100 of FIG. 6A with the window 314 positioned on the housing 302 in a differing position with respect to that of FIG. 6A, but with the same wedge, or quadrant, configuration. The window 314 may be positioned in various other positions along the first side wall 104, for example in the center or upper left and upper right corners.

FIG. 6C illustrates an additional configuration of IMD 100 having a window 414. The window 414 includes a semi ovular shape, with a linear edge 438 extending between the ends of a curved portion 440. FIG. 6D illustrates an additional embodiment of IMD 100 comprising a window 514 wherein the window 514 includes a generally rectangular shape and is positioned generally centrally with respect to a width of the first side wall 204 of the IMD 100. While illustrated as having a rectangular configuration, the window 514 may also comprise a square shape. Further, FIG. 6E illustrates the implantable medical device 100 of FIG. 1 with the window 114 having a circular configuration, similar to that of the window 114 shown in at least FIGS. 1 and 2. In various embodiments, the window 114 is shaped such that it has rounded corners for at least reducing stress risers, fracture and/or interface issues between the window 114 and remaining components of the IMD 100. In any of the embodiments illustrated herein, positioning of the window 114 may be varied along the first and/or second side walls 104, 106 of the housing 102. Further, the sizing of window 114 may be varied to have a smaller or larger surface area than those shown in the present embodiments. Additionally, other shapes of window 114 may be incorporated, for example, but without limitation, triangular, pentagonal, octagonal, or generally irregular shapes. While described with reference to the window 114, the disclosure herein applies to any of the windows of FIGS. 1-6E.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An implantable medical device comprising:
a hermetically sealed housing including at least a first window forming a portion of housing and configured for wireless transfer of an external power signal therethrough;
an antenna disposed within the housing at a position such that the antenna can receive the external power signal through the window; and
circuitry disposed within the housing and operatively coupled to the antenna.

2. The implantable medical device of claim 1, wherein the housing comprises first and second side walls and a peripheral wall extending therebetween, and wherein the first window forms a portion of the first side wall.

3. The implantable device of claim 2, further comprising a braze ring positioned between the first window and the first side wall.

4. The implantable medical device of claim 1, wherein the housing further includes a second window, wherein the first and second windows are disposed on opposite sides of the housing, and wherein the antenna is disposed within the housing at a position such that the antenna can receive an external power signal through first window.

5. The implantable medical device of claim 4, wherein the housing includes first and second side walls and a peripheral wall extending therebetween, and wherein the first window forms a portion of the first side wall, and the second window forms a portion of the second side wall, and further wherein the antenna is disposed within housing at a position such that the antenna can receive an external power signal through the first window.

6. The implantable device of claim 1, wherein the first window has one of a circular, semi-circular, and ovular shape.

7. The implantable device of claim 1, wherein the first window is formed of a non-metallic material.

8. The implantable device of claim 7, wherein the first window is formed from a ceramic.

9. The implantable medical device of claim 1, wherein the antenna is configured as a planar antenna.

10. The implantable medical device of claim 1, further comprising a battery operatively coupled to the antenna and further operatively coupled to the circuitry for providing power thereto.

11. The implantable medical device of claim 1, wherein the antenna receives the external power signal through the window to directly power the circuity of the implantable medical device.

12. The implantable medical device of claim 1, wherein the antenna is positioned adjacent a ferrite layer.

13. The implantable medical device of claim 12, wherein the ferrite layer is positioned adjacent a copper layer.

14. The implantable medical device of claim 1, wherein the antenna is positioned adjacent a copper layer.

15. An implantable medical device comprising:
a hermetically sealed housing defining an interior chamber and including
at least one window forming a portion of the housing and configured for signal transfer between an external device and the interior chamber;
a first antenna disposed within the interior chamber at a position such that the first antenna can receive an external power signal through one of the window;
a second antenna disposed within the interior chamber at a position such that the second antenna can receive and transmit signals through the at least one window; and
circuitry disposed within the interior chamber and operatively coupled to the first antenna.

16. The implantable medical device of claim 15, wherein the housing comprises first and second side walls and a peripheral wall extending therebetween, and wherein the first window forms a portion of the first side wall.

17. The implantable medical device of claim 16, wherein the at least one window further includes a second window forming a portion of the second side wall.

18. A method of making an implantable medical device, the method comprising:
forming a housing having a first side wall, a second side wall and a peripheral wall therebetween, wherein the first side wall includes a window configured for wireless transfer of an external power signal therethrough;
positioning an antenna within the housing such that the antenna can receive the external power signal through the window; and
positioning circuitry within the housing, the circuitry operatively coupled to the antenna.

19. The method of claim 18, further comprising positioning a battery within the housing, the battery being operatively coupled to the antenna and further operatively coupled to the circuitry for providing power thereto.

20. The method of claim 18, wherein the antenna is configured to receive the external power signal through the window to directly power the circuity of the implantable medical device.
